(19) United States
(12) Patent Application Publication
(10) Pub. No.: US 2026/0081040 A1
(43) Pub. Date: Mar. 19, 2026

(54) System and Method for Assisting a Participant

(71) Applicant: Blackcoffee Brand Expression Inc., Charlestown, MA (US)

(72) Inventors: Mark A. Gallagher, Charlestown, MA (US); Laura E. Gallagher, Charlestown, MA (US)

(21) Appl. No.: 19/327,992

(22) Filed: Sep. 12, 2025

Related U.S. Application Data
(60) Provisional application No. 63/694,761, filed on Sep. 13, 2024.

(51) Int. Cl. G16H 80/00; G06T 13/40; G16H 50/20

(52) U.S. Cl. CPC G16H 80/00; G06T 13/40; G16H 50/20

(57) ABSTRACT

A system and corresponding method assist a participant. The system comprises an avatar and a participant interaction and monitoring system (PIMM) that includes an engagement engine (EE), communicatively coupled to the avatar, and a trusting relationship (TR) dataset that supports the EE by storing TR content associated with a TR between the avatar and participant and developing a dynamic personality profile that supports effective communications between the EE, via the avatar, and the participant. The effective communications represent shared comprehension of a subject matter between the participant and EE. The PIMM includes an application-specific database that supports the EE by providing application-specific content, germane to the subject matter. The EE uses a combination of the application-specific content and the TR content to cause the avatar to interact with the participant in a manner that draws participant input from the participant to the EE via the avatar due to the TR.

### RELATED APPLICATION

This application claims the benefit of U.S. Provisional Application No. 63/694,761, filed on Sep. 13, 2024. The entire teachings of the above application(s) are incorporated herein by reference.

### BACKGROUND

Individuals often face challenges in assessment, monitoring, training, and intervention across diverse domains, including but not limited to cognitive health, education, and professional training. To address such challenges, ongoing evaluation, timely feedback, and effective adaptation are useful to ensure desired outcomes.

### SUMMARY

Current approaches typically provide unidirectional solutions that either deliver content or collect responses, but rarely both, in a coordinated manner. As a result, existing systems fail to achieve the bidirectional interaction useful for effective assessment, personalized training, and adaptive intervention across healthcare, educational, and professional contexts. In contrast to existing systems, an example embodiment of a system disclosed herein provides a “two-way system” with bidirectional functionality across multiple application domains.

According to an example embodiment, an assistive system comprises an avatar and a participant interaction and monitoring system (PIMM). The PIMM includes an engagement engine communicatively coupled to the avatar. The PIMM further includes a trusting relationship dataset configured to support the engagement engine by (i) storing trusting relationship content associated with a trusting relationship between the avatar and a participant and (ii) developing a dynamic personality profile that supports effective communications between the engagement engine, via the avatar, and the participant. The effective communications represent shared comprehension of a subject matter between the participant and the engagement engine. The PIMM further includes an application-specific database configured to support the engagement engine by providing application-specific content, germane to the subject matter for which the assistive system is being used to benefit the participant. The engagement engine is configured to use a combination of the application-specific content and the trusting relationship content to cause the avatar to interact with the participant in a manner that draws participant input from the participant to the engagement engine via the avatar due to the trusting relationship.

The trusting relationship dataset may include or may be developed as a function of recorded information of interactions between the engagement engine, via the avatar, and the participant.

The trusting relationship dataset may be a) accessible by a caregiver, b) recorded information developed as a result of interactions between the avatar, participant, and optionally a third party, or a) and b).

The engagement engine may be configured to identify an inconsistency between a response from the participant, in reaction to a participant-focused question or command from the avatar, and an expected response based on content in the trusting relationship dataset and content in the application-specific database.

The engagement engine may be further configured to recognize reduced intelligibility of a response from the participant, in reaction to a participant-focused question or command from the avatar, compared to intelligibility of a past response to the same or similar participant-focused question or command.

The avatar may be a likeness of a character human, animal, or fictional creature for non-limiting examples.

The trusting relationship dataset may include content that is supplied by historical information associated with the participant or a person or organization associated with the participant or captured through interaction of the participant and the avatar. For non-limiting example, the content may include at least a subset of: personal information of the participant, physiological information of the participant, psychological information of the participant, personality information of the participant, sociocultural information of the participant, behavioral information of the participant, cognitive information of the participant, emotional information of the participant, personal information of a person trusted by the participant or a person known by the participant, common life experiences, common connections to the person, human relationship connections; experiences in common with the person; and interests of the participant or the person trusted by the participant, the interests including at least one of: entertainment, politics, vocation, avocation, and religion, for non-limiting examples.

The application-specific database may include content that is at least a subset of: demographic information, financial information, education, vocation and occupation and professional information, health and mental information, preferences and interests, and location or regional culture information for non-limiting examples.

The engagement engine may be further configured to be activated by the participant or a third party.

The engagement engine may be further configured to employ artificial intelligence (AI), machine learning, or combination thereof.

The assistive system may further comprise a human-machine interface configured to represent the avatar to the participant and to capture interactions between the participant and the engagement engine.

At least one input to the human-machine interface may represent an active state of the participant.

The engagement engine may be further configured to interpret at least one input to the human-machine interface from the participant as an age-related cognitive disorder, a traumatic brain injury, a cognitive change at any point in time relative to an earlier point in time that is earlier relative to a current time at which the at least one input is input to the assistive system, or an earlier average level of cognitive function or learning performance, the earlier average level of cognitive function or learning performance being earlier relative to the current time, for non-limiting examples.

The engagement engine may be further configured to interpret at least one input to the human-machine interface from the participant based on a level of achievement of the participant within an application the assistive system is being used to benefit the participant, the level of achievement associated with educational achievement or training achievement. The application the assistive system is being used to benefit the participant may be selected from a group that may include medical evaluation, psychological evaluation, sales training and education, training, education, relationship training, school testing, proxy for third party, interview, and drug evaluation for non-limiting examples.

According to another example embodiment, a method for diagnosing, monitoring, and improving cognitive health issues in a participant using an avatar comprises collecting real-time data on the participant's behavior and cognitive responses through interactive sessions with the avatar used. The method further comprises analyzing the real-time data collected. The analyzing uses artificial intelligence (AI) computer-implemented methods. The AI computer-implemented methods include machine learning and anomaly detection techniques used to identify abnormalities, assess cognitive function, and provide cognitive exercises. The method further comprises dynamically generating and conducting cognitive tests and exercises during the interactive sessions based on the participant's inputs and performance to gather additional diagnostic and therapeutic data. The method further comprises providing comprehensive diagnostic and therapeutic information, including visualizations and trend analyses, to healthcare providers for timely intervention, personalized treatment, and cognitive recovery plans for the participant.

According to another example embodiment, a system for monitoring and improving cognitive health of a participant comprises an avatar modeled after a trusted figure. The avatar is created using advanced AI techniques for engaging and personalized interactive sessions with the participant. The system further comprises high-resolution audio and video interfaces for collecting real-time behavioral and cognitive data during the interactive sessions. The system further comprises an AI engine employing machine learning, natural language processing (NLP), sentiment analysis methods, and cognitive exercise generators for analyzing collected data to detect cognitive health issues, assess cognitive function, and promote cognitive improvement. The system further comprises a secure communication module for transmitting encrypted diagnostic and therapeutic information and integrating with electronic health record (EHR) systems to facilitate seamless data sharing with healthcare providers.

It should be understood that example embodiments disclosed herein can be implemented in the form of a method, apparatus, system, or computer readable medium with program codes embodied thereon.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a block diagram of an example embodiment of an assistive system.

FIGS. 2A-C are a flow diagrams of an example embodiment of a method that may be performed by the assistive system of FIG. 1.

FIG. 3 is a flow diagram of an example embodiment of a method for diagnosing, monitoring, and improving cognitive health issues in a participant using an avatar.

FIG. 4 is a block diagram of an example embodiment of a system for monitoring and improving cognitive health of a participant.

FIG. 5 is a block diagram of an example of the internal structure of a computer in which various embodiments of the present disclosure may be implemented.

### DETAILED DESCRIPTION

A description of example embodiments follows.

An example embodiment described herein includes a system, and corresponding method, that may leverage advanced computational technologies, including artificial intelligence (AI), machine learning, and other computer-assisted reasoning, in combination with an avatar that may represent an individual known to a participant for non-limiting example, or may otherwise be modeled after a real person or a familiar figure, as another non-limiting example, for diagnosing, monitoring, and improving cognitive health abnormalities or cognitive changes of the participant at any point in time in a health-related context, or for training and education in other contexts.

According to an example embodiment, the avatar may be caused by the system and method to interact with the participant through realistic video and audio representations, for non-limiting examples. The system may monitor cognitive behavior by detecting abnormalities, conducting cognitive tests, providing exercises for improvement, and adapting training and educational methods to the participant's specific needs. The system may analyze interactions in real-time to detect the participant's current state, monitor changes and adapt behavior for applications, such as cognitive decline and traumatic brain injury (TBI) for non-limiting examples, provide diagnostic and therapeutic information to healthcare providers and/or improving training and education outcomes for non-limiting examples. Additional example embodiments may be directed to sales training, education training, mental health monitoring, poker playing, couples'therapy, drug testing, chess playing, monitoring addiction behaviors, and/or improving training and education outcomes for non-limiting examples.

Current technologies, such as wearables and mobile apps, focus on general mental health monitoring but do not offer a personalized approach leveraging emotional and psychological benefits of interacting with a familiar figure to a participant engaging with the technology. This lack of personalization for the participant can result in the participant's lower engagement and adherence rate, limiting effectiveness of solutions of the current technologies.

An example embodiment disclosed herein may address this gap by creating an advanced computational technology driven avatar modeled after a familiar figure to the participant, such as a real person for non-limiting example, enhancing engagement, comfort, and mental well-being during interactions, where the advanced computational technology may be artificial intelligence (AI), machine learning (ML), or other computer-assisted reasoning AI system used to drive the avatar. In a healthcare application, for example, the familiar figure, having a familiar appearance and/or voice presented by the avatar, can help reduce feelings of loneliness and isolation, which are common among individuals with cognitive health issues and can contribute to cognitive decline or hinder recovery. Additionally, the avatar may monitor cognitive function, conduct specific tests, and provide cognitive exercises to facilitate improvement. By engaging the participant in a more natural and comforting manner, an example embodiment aims to improve accuracy and efficiency of diagnosing, monitoring, and treating cognitive health issues, including those resulting from TBI.

Accordingly, an example embodiment disclosed herein may provide a system and method for diagnosing, monitoring, and improving cognitive health issues in participants using an AI-powered avatar in accordance with example embodiments disclosed herein. The avatar may be created by digitizing visual and audio samples from a loved one or trusted figure for non-limiting examples. This avatar may interact with the participant through realistic video and audio interfaces, monitoring conversations for signs of cognitive health issues. A participant interaction and monitoring manager (PIMM) may be communicatively coupled to the avatar and may cause the avatar to interact with the participant and may continuously or continually analyze verbal cues and/or visual cues of the participant to detect signs of impairment or progress of a given condition for non-limiting examples. Upon detecting potential issues, the avatar may be caused by the PIMM to ask targeted questions, request tasks to be performed by the participant to assess the participant's cognitive abilities and may provide cognitive exercises to the participant to promote recovery for non-limiting examples.

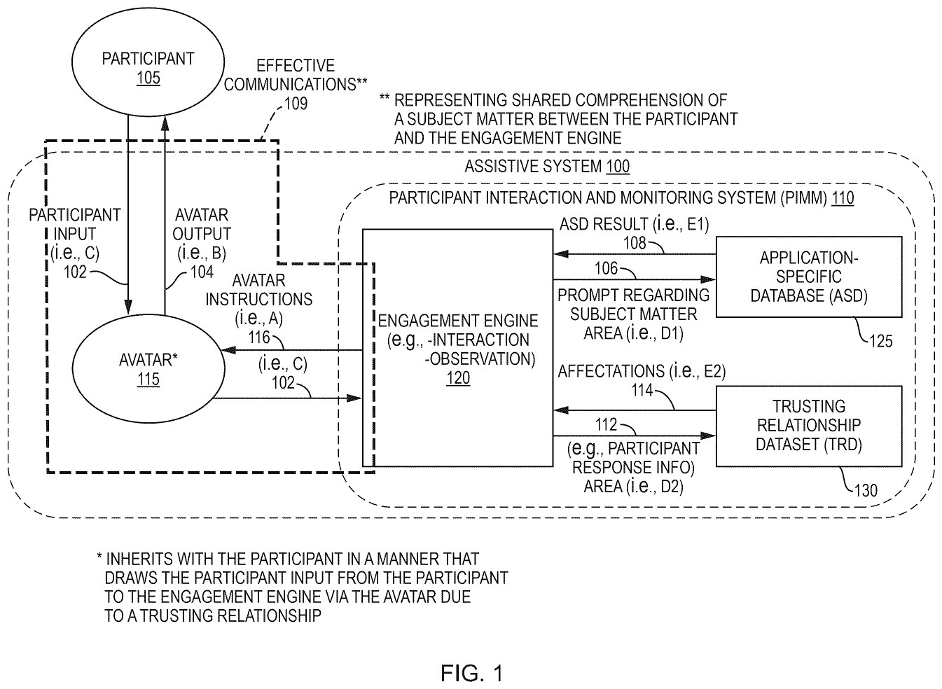

The interactions between the avatar and the participant may be recorded and analyzed in non-real-time, or data representing the interactions may be analyzed in real-time to provide diagnostic and therapeutic information to a healthcare provider. The system may include robust data security measures to ensure privacy and compliance with relevant health regulations. An example embodiment of such a system is disclosed below with reference to FIG. 1.

FIG. 1 is a block diagram of an example embodiment of an assistive system 100 that comprises an avatar 115 and a participant interaction and monitoring system (PIMM) 110. The PIMM 110 includes an engagement engine 120 communicatively coupled to the avatar 115. The PIMM 110 further includes a trusting relationship dataset 130 configured to support the engagement engine 120 by (i) storing trusting relationship content (not shown) associated with a trusting relationship between the avatar 115 and a participant 105 and (ii) developing a dynamic personality profile (not shown) that supports effective communications 109 between the engagement engine 120, via the avatar 115, and the participant 105. The effective communications 109 represent shared comprehension of a subject matter between the participant 105 and the engagement engine 120. The PIMM 110 further includes an application-specific database 125 configured to support the engagement engine 120 by providing application-specific content (not shown), germane to the subject matter for which the assistive system 100 is being used to benefit the participant 105. The engagement engine 120 is configured to use a combination of the application-specific content and the trusting relationship content to cause the avatar 115 to interact with the participant 105 in a manner that draws participant input 102 from the participant to the engagement engine 120 via the avatar 115 due to the trusting relationship.

The avatar 115 may be a likeness of a character human, animal, or fictional creature for non-limiting examples. The trusting relationship dataset 130 may include or may be developed as a function of recorded information of interactions between the engagement engine 120, via the avatar 115, and the participant 105. The trusting relationship dataset 130 may be a) accessible by a caregiver (not shown), b) recorded information developed as a result of interactions between the avatar 115, participant 105, and optionally a third party (not shown), or a) and b).

The engagement engine 120 may be configured to identify an inconsistency between a response from the participant 105, such as the participant input 102, in reaction to a participant-focused question or command from the avatar 115, such as the avatar output 104, and an expected response (not shown) based on content in the trusting relationship dataset 130 and content in the application-specific database 125. It should be understood that the participant input 102 is not limited to being a response and may, for example be a query. It should also be understood that the avatar output 104 is not limited to being a participant-focused question or command and could, for non-limiting example, be a response to the participant input 102.

The engagement engine 120 may be further configured to recognize reduced intelligibility of a response (e.g., participant input 102) from the participant 105, in reaction to a participant-focused question or command (e.g., avatar output 104) from the avatar 115, compared to intelligibility of a past response to the same or similar participant-focused question or command.

The trusting relationship dataset 130 may include content that is supplied by historical information associated with the participant 105 or a person or organization associated with the participant 105 or captured through interaction of the participant 105 and the avatar 115. For non-limiting example, the content may include at least a subset of: personal information of the participant 105, physiological information of the participant 105, psychological information of the participant 105, personality information of the participant 105, sociocultural information of the participant 105, behavioral information of the participant 105, cognitive information of the participant 105, emotional information of the participant 105, personal information of a person trusted by the participant 105 or a person known by the participant 105, common life experiences, common connections to the person, human relationship connections; experiences in common with the person; and interests of the participant 105 or the person trusted by the participant 105, the interests including at least one of: entertainment, politics, vocation, avocation, and religion for non-limiting examples.

The application-specific database 125 may include content that is at least a subset of: demographic information, financial information, education, vocation and occupation and professional information, health and mental information, preferences and interests, and location or regional culture information for non-limiting examples.

The engagement engine 120 may be further configured to be activated by the participant 105 or a third party (not shown). The engagement engine 120 may be further configured to employ artificial intelligence (AI), machine learning, or combination thereof.

The assistive system 100 may further comprise a human-machine interface (not shown) configured to represent the avatar 115 to the participant 105 and to capture interactions between the participant 105 and the engagement engine 120. At least one input to the human-machine interface may represent an active state of the participant 105. The engagement engine 120 may be further configured to interpret at least one input to the human-machine interface from the participant 105 as an age-related cognitive disorder, a traumatic brain injury, a cognitive change at any point in time relative to an earlier point in time that is earlier relative to a current time at which the at least one input is input to the assistive system, or an earlier average level of cognitive function or learning performance, the earlier average level of cognitive function or learning performance being earlier relative to the current time, for non-limiting examples.

The engagement engine 120 may be further configured to interpret at least one input to the human-machine interface from the participant 105 based on a level of achievement of the participant 105 within an application the assistive system 100 is being used to benefit the participant 195, the level of achievement associated with educational achievement or training achievement. The application the assistive system 100 is being used to benefit the participant 105 may be selected from a group including: medical evaluation, psychological evaluation, sales training and education, training, education, relationship training, school testing, proxy for third party, interview, and drug evaluation for non-limiting examples.

Further technical details are disclosed below.

Continuing with reference to FIG. 1, an operational and data flow of the assistive system 100 is shown. The avatar 115 is arranged to interact with the participant 105, and the PIMM 110 causes the avatar 115 to interact with the participant 105 in a manner that is appropriate and trustworthy to the participant 105.

The avatar 115 may be a video or graphical representation in the form of a person that the participant 105 knows or may be in the form of a figure with whom the participate 105 is familiar/trusts for non-limiting examples. For non-limiting examples, the avatar 115 may be a video display, audio presentation, graphical display with two-dimensional (2D) or three-dimensional (3D) representation or illustration, light display, or any other interactive representation or medium that is capable of interacting with the participant 105 in a manner that the participant 105 has a trusting relationship with the avatar 105 such that meaningful information may be gleaned by the PIMM 110.

The assistive system 100 may be operated by a healthcare provider (not shown) in an example embodiment in which the participant 105 is a patient or otherwise a person who is in need of care in the form of diagnostic observation or treatment. For example, individuals often face cognitive health issues for which ongoing monitoring, timely diagnosis, and effective interventions are useful to ensure proper care. These health issues can lead to a decline in quality of life, increased healthcare costs, and a significant burden on caregivers. In other applications and for non-limiting examples, a person (not shown) operating the assistive system 100 may be working in a different field of endeavor, such as: training the participating 105 to gain new or different career skills (e.g., sales); educating the participant 105 in games, such as poker playing or chess for non-limiting examples; providing therapy to the participant 105 or multiple participants (not shown) in couples therapy or group therapy; monitoring a drug test for the participant 105 involved with drug testing; or, serving in a pharmacist capacity in which the assistive system 100 may be used for monitoring the participant 105 for addiction behavior during a prescription drug refill for non-limiting examples.

The application-specific database 125 and trusting relationship dataset 130 may be referred to interchangeably herein as an ASD 125 and TRD 130, respectively. The engagement engine 120 may be configured to provide prompts (106, 112) to the ASD 125 and TRD 130 in a manner suitable for each. The ASD 125 and TRD 130 may be configured to return respective information (108, 114) for the engagement engine 120 to present instructions to the avatar 115 in a manner that causes the avatar 115 to interact with the participant 105 in a manner that is appropriate for the application (i.e., activity in which the participant 105 is participating), as well as consistent with the trusting relationship that the participant 105 has with the person or other figure represented by the avatar 115.

Continuing with reference to the PIMM 110, the engagement engine 120 may be used for interaction and observation of the participant 105 via the avatar 115. The interactions may be produced in the form of avatar instructions 116 (i.e., A), and the observations may be received in the form of participant input 102 (i.e., C) from the participant 105. The engagement engine 120 may be configured to learn what interactions are appropriate for the application in which the participant 105 is participating by way of a prompt 106 (e.g., regarding subject matter area D1) made to the ASD 125. The ASD 125 may be configured to provide an ASD result 108 (i.e., E1) to the engagement engine 120. The engagement engine 120, serially or in parallel with sending the prompt 106 (i.e., D1) to the ASD 125, may be configured to provide participant response information 112 (i.e., D2) to the TRD 130 and, in turn, receives affectations 114 (i.e., E2) to combine with the ASD result 108 (i.e., E1) to form the avatar instructions 116 (i.e., A). The avatar instructions 116 (i.e., A) may be provided to the avatar 115 which, in turn, may be configured to produce avatar output 104 (i.e., B) to the participant 105, such as participant-focused questions/commands for non-limiting example.

Thus, an example embodiment of a workflow in the assistive system 100 can be recognized for non-limiting example as including the following: the engagement engine 120 provides avatar instructions 116 (i.e., A) to the avatar 115. The avatar 115 presents avatar output 104 (i.e., B) to the participant 105, such as participant-focused questions/commands for non-limiting example. The participant 105 reacts to the avatar output 104 (i.e., B), which may be captured in the form of the participant input 102 (i.e., C) by the avatar 115, which may forward the participant input 102 (i.e., C) to the engagement engine 120. The engagement engine 120 may, responsively, send a prompt 106 (i.e., D1) regarding a subject matter area to the ASD 125, which may return an ASD result 108 (i.e., E1) to the engagement engine 120. The engagement engine 120 may also send participant response information 112 (i.e., D2) to the TRD 130, which may return the affectations 114 (i.e., E2). It should be understood that a workflow in the assistive system 100 may begin with the avatar 115 receiving the participant input 102 (i.e., C).

It should be understood that the ASD 125 may be a locally maintained database that has significant information about the application in which the participant 105 is participating. Alternatively, the ASD 125 may have connectivity to online databases (not shown) or search engines (not shown), thereby enabling access to information worldwide for a particular application (e.g., cognitive observation or education) that is useful to the participant 105.

It should also be understood that the TRD 130 may have short-term and/or long-term memory such that participant response information 112 (i.e., D2) may be captured and utilized by the TRD 130 to provide the affectations 114 (i.e., E2) that may, in turn, be employed by the engagement engine 120 to provide custom and appropriate responses for the avatar instructions 116 (i.e., A). Through memory of past interactions between the participant 105 and avatar 115 and usage of that memory in the form of the affectations 114 (i.e., E2) during future interactions, the participant 105 will maintain or increase trust over time with the avatar 115.

The TRD 130 may be configured to provide the affectations 114 (i.e., E2) that enable the engagement engine 120 to generate avatar instructions 116 (i.e., A) that may be commands that cause the avatar 115 to demonstrate empathy, humor, or other personalized effects known to aid in the participant's 105 acceptance, appreciation, and trust of the avatar 115.

As should be understood from the foregoing, the ASD 125 includes extensive information around the reason for the participant's 105 engagement, and the TRD 130 records participant response information 112 (i.e., D2) and responds with the affectations 114 (i.e., E2) to customize the avatar's 115 interaction with the participant 105.

According to an example embodiment, the assistive system 100 may work in two ways. Like a motor that can also work as a generator, the assistive system 100 may operates in two modes. Both use the same parts, but in a different order. In an education mode, the assistive system 100 may start by giving information to the subject (push), such as the avatar output 104 output to the participant 105, then collecting a response (pull), such as the participant input 102. In a diagnostic monitoring mode, the assistive system 100 may start by collecting information from the subject (pull), such as by receiving the participant input 102, and then sending targeted probes (push) that may be included in the avatar output 104.

### Education (Push First, Pull Second)

In an education mode, the engagement engine 120 may present content from the application-specific database 125 through the avatar 115. The engagement engine 120 may adapt a teaching method and style using the trusting relationship dataset 130 and past interactions in the engagement engine 120 application-specific database 125. After presenting the content, the engagement engine 120 may collects the subject's responses represented by the participant input 102 in real time. The engagement engine 120 may check these responses against expected content, compare them with the trusting relationship dataset 130, and measure clarity against the subject's normal pattern. The engagement engine 120 may then adjust as needed, such as changing tone, methods, or examples, and records a result. Over time, the engagement engine 120 may adapt the methods and examples for the same module, so the lesson is presented in a manner that matches the subject's intellect and learning style.

### Diagnostic Monitoring (Pull First, Push Second)

In a diagnostic monitoring mode, the engagement engine 120 may start by collecting input from the subject, such as the participant input 102 that may represent symptoms or signs of an event listed in the application-specific database 125. Using this input and the subject's history in the engagement engine 120 database, the engagement engine 120 may choose and present one or more probes through the avatar 115 and record the subject's responses represented by the participant input 102. The monitoring system, that is, the PIMM 110, may check these responses for inconsistency and reduced clarity compared to the trusting relationship dataset 130 and the engagement engine database, that is, the application-specific databased 125. If certain limits are reached, the assistive system 100 may send a notification (not shown) to a third party. All interactions may be stored in the engagement engine 120 database, such as the application-specific database 125.

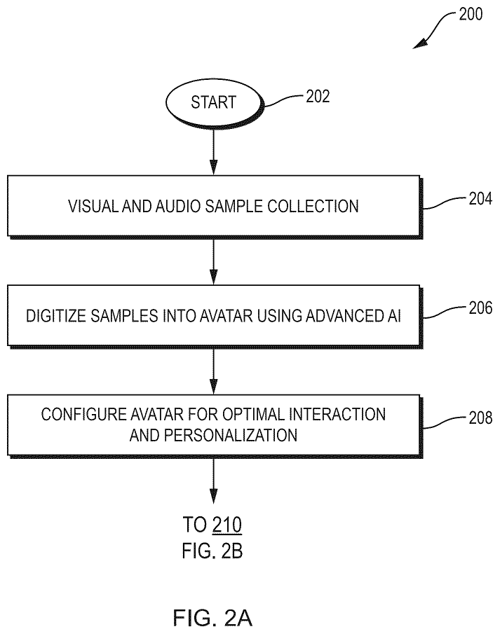

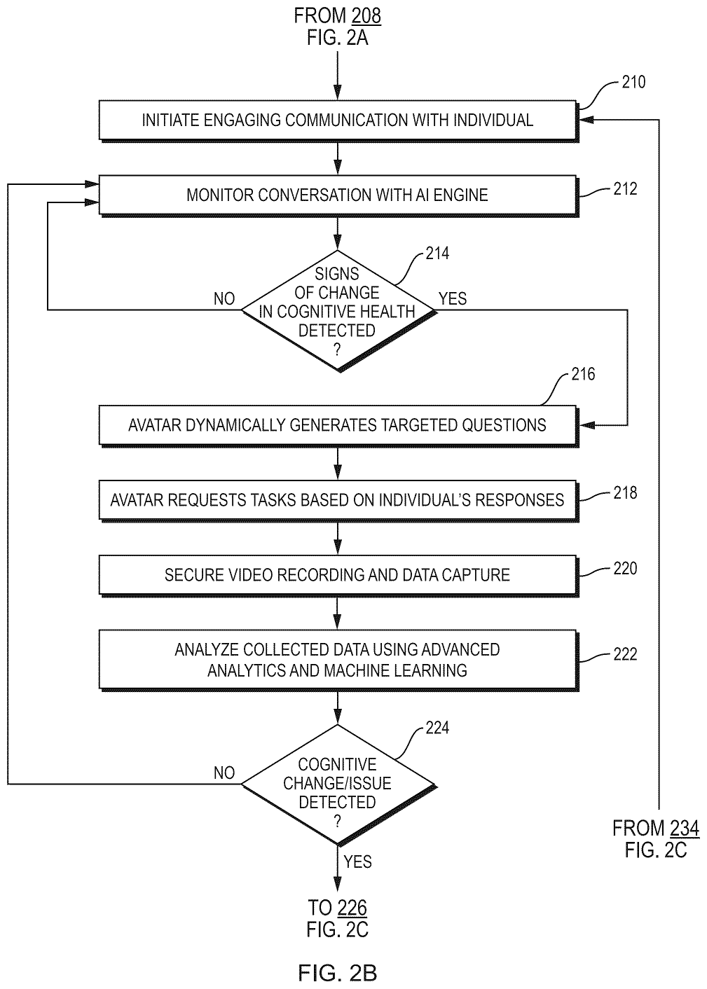

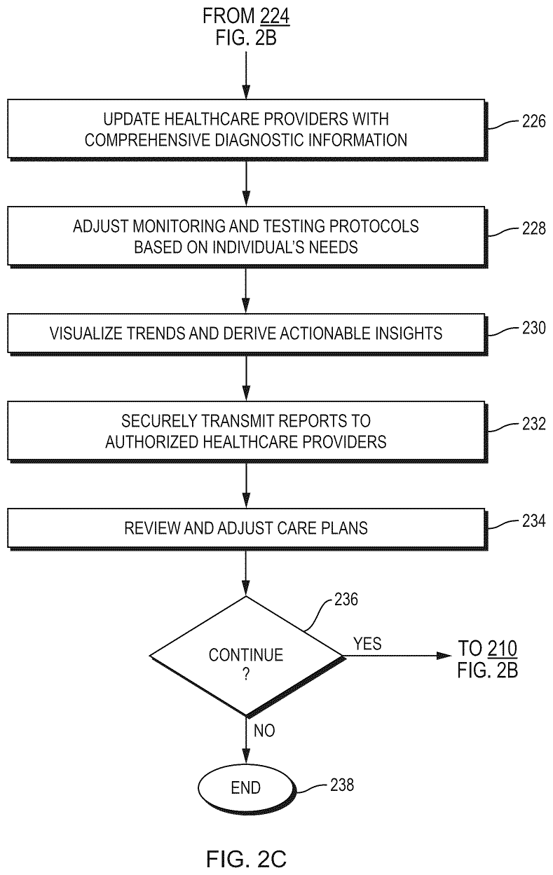

FIG. 2A is a flow diagram of an example embodiment of a method 200 that may be performed by the assistive system 100 of FIG. 1, disclosed above.

FIGS. 2B and 2C are a continuation of FIG. 2A. With reference to FIG. 1 and FIGS. 2A-C, the method 200 begins (202) and may comprise performing visual and audio sample collection (204). For example, a loved one or trusted figure may provide visual and audio samples. These samples may be digitized by the PIMM 110 to create a realistic avatar resembling a person for non-limiting example. The method 200 may comprise digitizing samples, by the PIMM 110, into the avatar 115 using AI (206), such as by using advanced AI computer-implemented methods. For example, the collected samples may be processed using advanced AI computer-implemented methods to generate a highly realistic and interactive avatar. The method 200 may comprise configuring the avatar 115 by the PIMM 110 for optimal interaction and personalization (208). For example, the avatar's settings and parameters can be configured for optimal interaction based on an individual's needs. The method 200 may comprise, by the avatar 115, initiating engaging communication with the individual (e.g., the participant 105) (210). The engagement engine 120 may be referred to interchangeably herein as an AI engine 120. The method 200 may further comprise monitoring conversion with the AI engine 120 (212). For example, the avatar 115 may communicate with the participant 105 through high-resolution video and clear audio interfaces, simulating a face-to-face conversation. The interaction may leverage the familiar appearance and voice of the avatar 115 to reduce loneliness and promote mental well-being. The monitoring may include employing natural language processing (NLP) and sentiment analysis techniques to ensure engaging and context-appropriate conversations. The AI engine 120 may monitor the conversation in real-time, analyzing verbal and non-verbal cues to detect signs of cognitive health issues. Advanced anomaly detection techniques may be used to identify behavioral abnormalities and assess cognitive function through natural dialogue.

The method 200 may further comprise checking for signs of change in cognitive health (214). If no signs of change are detected, the method 200 may again monitor conversation with the AI engine (212). If, however, no signs of change are detected at (214), the method 200 may comprise, employing the ASD 125 to cause the avatar 115 to dynamically generate targeted questions (216). For example, upon detecting potential cognitive health issues, the avatar 115 may dynamically generate targeted questions (216), request tasks based on the individual's responses (218), and provide cognitive exercises based on the individual's responses and performance. These interactions may be designed to probe various aspects of cognitive function and promote cognitive recovery. Adaptive computer-implemented methods may be employed to adjust the difficulty and nature of the questions, tasks, and exercises based on the individual's real-time performance.

The method 200 may comprises secure video recording and data capture (220). For example, interactions may be securely video recorded and relevant data may be captured for analysis and stored in the TRD 130. The method may comprise analyzing collected data using advanced analytics and machine learning (222). For example, the AI engine 120 may employ advanced data analytics to detect subtle changes and assess the rate of cognitive decline or improvement over time. The method 200 may check (224) for whether a cognitive change/issue has been detected. If no, the method may may again monitor conversation with the AI engine (212). If, however, the check (224) detects there is a cognitive change/issue, the method 200 may proceed to perform real-time analysis and reporting.

For example, the analyzed data may be compiled into comprehensive diagnostic and therapeutic reports, which include visualizations, trend analyses, and actionable insights for non-limiting examples. These reports may be securely transmitted to authorized healthcare providers, enabling timely intervention and personalized treatment plans. The system integrates seamlessly with existing electronic health record (EHR) systems. As such, the method 200 may may comprise updating healthcare providers with comprehensive diagnostic information (226), adjust monitoring and testing protocols based on the individual's needs (228), visualize trends and derive actionable insights (230), and securely transmit reports to authorized healthcare providers (232). The method may further comprise reviewing and adjusting care plans (234) and check (236) for whether to continue. If the decision at (236) is not to continue, the method thereafter ends (238) in the example embodiment. If, however, the decision at (236) is to continue, the method 200 may again initiate engaging communication with the individual (210).

As such, the method 200 may perform continuous monitoring and adjustment. The method 200 enables the assistive system 100 continuously monitor the individual's cognitive function over time, allowing for early detection of any changes or deterioration, as well as progress in recovery. According to an example embodiment, monitoring and testing protocols may be dynamically adjusted based on the latest analysis and the individual's specific needs. Robust data security measures, including encryption, access controls, and secure data storage, may be implemented to protect the individual's sensitive health information, thereby providing data security and privacy. The assistive system 100 may be designed to be fully compliant with relevant health data regulations, such as the General Data Protection Regulation (GDPR) and Health Insurance Portability and Accountability Act (HIPAA) for non-limiting examples.

An example embodiment disclosed herein may be employed in healthcare applications, such as the following healthcare applications for non-limiting examples:
Elderly care facilities for continuous mental health monitoring.
Remote health monitoring systems to support independent living for the elderly.
Integration with telehealth services to provide comprehensive mental health diagnostics and care.
Clinical trials and research on cognitive impairments in the elderly.
Employee mental health support in eldercare industries.

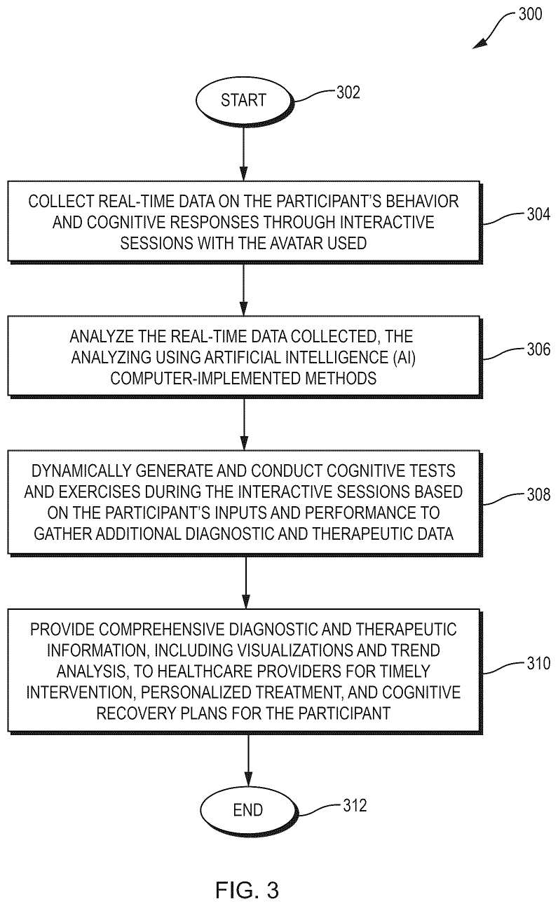

FIG. 3 is a flow diagram of an example embodiment of a method 300 for diagnosing, monitoring, and improving cognitive health issues in a participant using an avatar. The method 300 begins (302) and comprises collecting real-time data on the participant's behavior and cognitive responses through interactive sessions with the avatar used (304). The method 300 further comprises analyzing the real-time data collected (306). The analyzing may use artificial intelligence (AI) computer-implemented methods. The AI computer-implemented methods may include machine learning and anomaly detection techniques used to identify abnormalities, assess cognitive function, and provide cognitive exercises. The method 300 further comprises dynamically generating and conducting cognitive tests and exercises during the interactive sessions based on the participant's inputs and performance to gather additional diagnostic and therapeutic data (308). The method 300 further comprises providing comprehensive diagnostic and therapeutic information, including visualizations and trend analyses, to healthcare providers for timely intervention, personalized treatment, and cognitive recovery plans for the participant (310). The method thereafter ends (312) in the example embodiment.

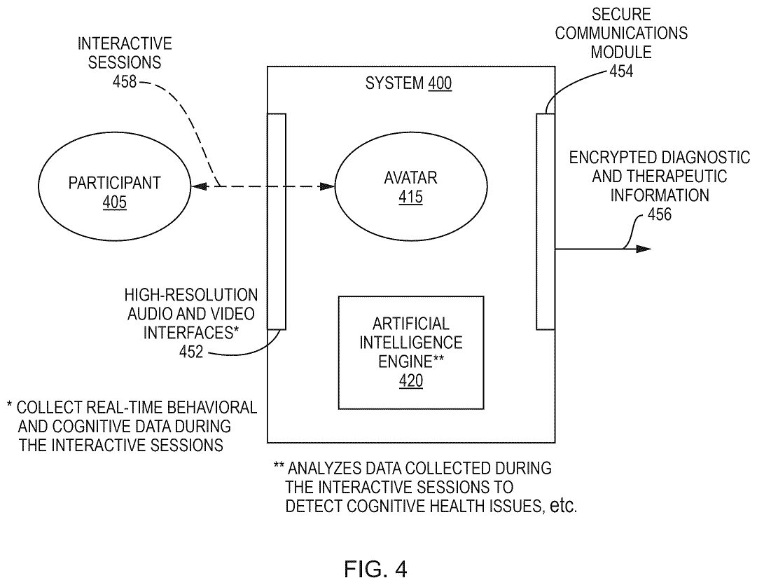

FIG. 4 is a block diagram of an example embodiment of a system 400 for monitoring and improving cognitive health of a participant 405. The system 400 comprises an avatar 415 modeled after a trusted figure (not shown). The avatar 415 is created using advanced AI techniques for engaging and personalized interactive sessions 458 with the participant 405. The system 400 further comprises high-resolution audio and video interfaces 452 for collecting real-time behavioral and cognitive data during the interactive sessions. The system 400 further comprises an AI engine 420 employing machine learning, natural language processing (NLP), sentiment analysis methods, and cognitive exercise generators for analyzing collected data to detect cognitive health issues, assess cognitive function, and promote cognitive improvement. The system 400 further comprises a secure communication module 454 for transmitting encrypted diagnostic and therapeutic information 456 and integrating with electronic health record (EHR) systems (not shown) to facilitate seamless data sharing with healthcare providers (not shown).

### EXEMPLIFICATIONS

### Echoes of Sarah

The rain tapped a steady rhythm against the windowpane. Inside the warm, softly lit room, a gentle hum from the heater underscored the conversation between two figures—one human, one digital.

“Robert, do you remember the summers we spent at the beach?” The avatar's voice, warm and soothing, carried through the room. Its digital face, an uncanny recreation of Robert's late wife, smiled gently. Robert, an 82-year-old retired engineer, squinted at the screen. His eyes, clouded with both age and memories, softened. “Of course, Sarah. Those were the best days, weren't they?”

The avatar, powered by an advanced AI system, nodded, its movements fluid and natural. “Yes, they were. Do you remember the name of the boat we rented?” Robert's brow furrowed slightly as he tried to recall. “It was . . . the Blue Marlin, wasn't it?” “That's right,” the avatar confirmed, its digital eyes twinkling. “You have a great memory, Robert.” The conversation between Robert and the avatar continued seamlessly. “How about we do a little exercise now?” the avatar suggested. “Let's play the word game we used to enjoy.” Robert's eyes lit up. “Sure, Sarah, let's do it.” The avatar began, “I'll say a word, and you say the first thing that comes to your mind. Ready?” Robert nodded. “Summer,” the avatar said. “Sunshine,” Robert responded quickly. “Ocean.” “Sand.”

As they continued, the AI in the avatar monitored Robert's responses, analyzing his reaction time, word associations, and emotional cues. Each data point was logged and sent to the central database for real-time analysis. The system was designed to detect any signs of cognitive decline and adjust the prompts accordingly. Today, the AI noted a slight reduction in Robert's vocabulary. Words he once used with ease now seemed to elude him, replaced by simpler terms. The AI adjusted the prompts for the next session to probe deeper into his cognitive abilities without overwhelming him.

The session concluded with the avatar praising Robert. “You did great today, Robert. I'll see you tomorrow, okay?” Robert smiled, a genuine warmth spreading across his face. “Okay, Sarah. Thank you.” As the screen dimmed, Robert sat back in his chair, feeling the comfort and companionship, the avatar provided. It wasn't just a tool for engagement; it was a sophisticated system designed to detect and diagnose cognitive impairments early.

Dr. Emily Carter was at her desk, reviewing patient reports when a notification popped up on her screen. She clicked it open and saw the alert from Robert's session. The AI had flagged a slight delay in his response to ‘ocean’ and a noticeable reduction in vocabulary, suggesting deeper cognitive issues. Emily reviewed the data collected by the AI and watched the video of the session. She was impressed by the system's analysis; while the hesitations were notable, she wondered if she could have detected the reduction in vocabulary herself. Not only did the system alert her to an issue that may indicate a decline in Robert's cognitive abilities that she may not have been able to detect herself, but it also allowed her to focus on reviewing the comprehensive diagnostic information and planning Robert's care with a more informed perspective.

The next day, Robert's avatar session included the new, AI-generated prompts designed to probe a bit deeper without overwhelming him. “Robert, can you describe the layout of the house we lived in by the beach?” the avatar asked. Robert's eyes twinkled as he began to recount the details, but there was a noticeable pause when describing the upstairs rooms. The AI logged the hesitation and the use of more generic terms instead of specific vocabulary he used to employ, providing valuable data for Emily. As the session ended, the avatar smiled warmly. “You did well today, Robert. I'll see you tomorrow.” Robert nodded, feeling a mix of nostalgia and satisfaction. The avatar, a digital echo of his past, had become a comforting presence in his life.

Emily reviewed the session's data, noting the slight hesitations and reduction in vocabulary. The technology combined machine learning, natural language processing, and advanced data analytics to provide real-time insights into Robert's cognitive health. Every interaction, every response, was a piece of a larger puzzle, helping doctors like Emily understand and address the complexities of mental decline. She knew they still had a long way to go, but with each session, they were one step closer to making a real difference. The AI's ability to adapt and refine its approach based on Robert's responses was crucial in providing personalized care.

In the quiet comfort of his home, Robert found solace in the digital presence of Sarah. Through that connection, a new wave of understanding and care emerged, heralding a future where technology and compassion walked hand in hand—just as Robert and Sarah once had.

Jane's Long Road

The sterile white walls of the rehab facility echoed with the faint sounds of distant conversations and the occasional beeping of medical equipment. Jane, a 45-year-old marketing executive, sat in her room, staring out the window at the meticulously manicured garden. It was a nice environment, but there was a difference between environment and atmosphere. Here, the atmosphere was one of loneliness, making her recovery feel even more arduous.

Jane had been in a car accident six months ago, suffering a traumatic brain injury. The impact had turned her world upside down, leaving her with cognitive challenges she never anticipated. The accident had robbed her of her sharpness, her confidence, and the sense of control she once had over her life. A soft chime interrupted her thoughts. She turned her attention to the screen on the desk where a familiar face appeared—an avatar resembling her best friend, Emma. The avatar smiled warmly.

“Hi, Jane! How are you feeling today?” the avatar asked. Jane's lips curled into a small smile. “Hi, Emma. I'm doing okay, I guess.” The avatar nodded. “That's good to hear. How about we do some puzzles to help with your recovery today?” Jane felt a surge of anticipation. “That sounds great.”

The avatar began with a series of puzzles designed to stimulate Jane's cognitive function. “Let's start with a word puzzle. Can you find a word that fits this definition: ‘A place where books are kept and read'?” Jane paused to think. “Library,” she said after a moment. “That's correct!” the avatar responded with a smile. “Now, let's try a logic puzzle. If you have three apples and you take away two, how many do you have?”

“You have two, the ones you took away.” “Exactly, Jane. Let's move on to a sequence puzzle. Imagine this sequence of numbers: 2, 4, 6, 8. What comes next?” “10,” Jane replied confidently. “Perfect! You're doing wonderfully,” the avatar praised. “Now, let's tackle a visual puzzle. I'm going to show you a pattern, and you tell me which shape completes it.” The screen displayed a series of geometric shapes. Jane examined them carefully before selecting the correct shape. “It's the circle,” she determined. “Well done, Jane. Your problem-solving skills are really improving,” the avatar said encouragingly. The session concluded with the avatar praising Jane. “You did a fantastic job today, Jane. Your progress is really impressive. I'll see you tomorrow, okay?” Jane smiled, feeling a sense of achievement. “Thanks, Emma. See you tomorrow.” As the screen dimmed, Jane felt accomplished. The avatar, a digital representation of her best friend, provided a comforting presence and a crucial tool in her recovery. It wasn't just about the exercises; it was about the connection, the feeling of not being alone.

Dr. David Jones, Jane's therapist, reviewed the data from Jane's session. The AI had flagged significant improvements in Jane's cognitive abilities. David watched the video of the session, impressed by Jane's progress. The AI's analysis captured subtle nuances in Jane's responses. It provided David with detailed insights, allowing him to tailor Jane's therapy plan more effectively. The technology enhanced the human touch, enabling a deeper understanding of Jane's needs.

The next day, the avatar greeted Jane with the same warmth. “Hi, Jane! Ready for another session?” Jane nodded, feeling excited and determined. As they worked through the puzzles, Jane felt more connected, more hopeful about her recovery. In the quiet of her room, Jane found solace and strength in the avatar's presence. Through their interactions, a new path to recovery emerged, blending technology and empathy to guide her towards a brighter future. Each session brought Jane closer to reclaiming her life, proving that even in a place where the atmosphere was one of loneliness, warmth and connection could still be found.

According to another example embodiment, a system for monitoring and improving cognitive health of a participant comprises an avatar modeled after a trusted figure. The avatar is created using advanced AI techniques for engaging and personalized interactive sessions with the participant. The system further comprises high-resolution audio and video interfaces for collecting real-time behavioral and cognitive data during the interactive sessions. The system further comprises an AI engine employing machine learning, natural language processing (NLP), sentiment analysis methods, and cognitive exercise generators for analyzing collected data to detect cognitive health issues, assess cognitive function, and promote cognitive improvement. The system further comprises a secure communication module for transmitting encrypted diagnostic and therapeutic information and integrating with electronic health record (EHR) systems to facilitate seamless data sharing with healthcare providers.

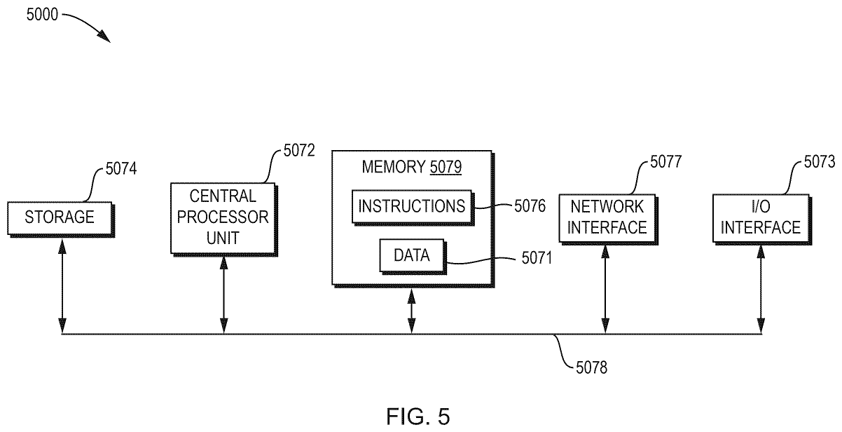

FIG. 5 is a block diagram of an example of an internal structure of a computer 5000 in which various embodiments of the present disclosure may be implemented. The computer 5000 contains a system bus 5078, where a bus is a set of hardware lines used for data transfer among the components of a computer or digital processing system. The system bus 5078 is essentially a shared conduit that connects different elements of a computer system (e.g., processor, disk storage, memory, input/output ports, network ports, etc.) that enables the transfer of information between the elements. Coupled to the system bus 5078 is an I/O device interface 5073 for connecting various input and output devices (e.g., keyboard, mouse, display monitors, printers, speakers, microphone, etc.) to the computer 5000. A network interface 5077 allows the computer 5000 to connect to various other devices attached to a network (e.g., global computer network, wide area network, local area network, etc.). Memory 5079 provides volatile or non-volatile storage for computer software instructions 5076 and data 5071 that may be used to implement embodiments (e.g., methods 200 and 300) of the present disclosure, where the volatile and non-volatile memories are examples of non-transitory media. Disk storage 5076 also provides non-volatile storage for the computer software instructions 2011 and data 5071 that may be used to implement embodiments (e.g., methods 200 and 300) of the present disclosure. A central processor unit 5072 is also coupled to the system bus 5078 and provides for the execution of computer instructions.

Example embodiments disclosed herein may be configured using a computer program product. Further example embodiments may include a non-transitory computer-readable medium that contains instructions that may be executed by a processor, and, when loaded and executed, cause the processor to complete methods described herein.

In addition, the elements described herein may be combined or divided in any manner in software, hardware, or firmware. If implemented in software, the software may be written in any language that can support the example embodiments disclosed herein. The software may be stored in any form of computer readable medium, such as random-access memory (RAM), read-only memory (ROM), compact disk read-only memory (CD-ROM), and so forth.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. An assistive system, comprising:
an avatar; and
a participant interaction and monitoring system (PIMM) including:
an engagement engine communicatively coupled to the avatar;
a trusting relationship dataset configured to support the engagement engine by (i) storing trusting relationship content associated with a trusting relationship between the avatar and a participant and (ii) developing a dynamic personality profile that supports effective communications between the engagement engine, via the avatar, and the participant, the effective communications representing shared comprehension of a subject matter between the participant and the engagement engine; and
an application-specific database configured to support the engagement engine by providing application-specific content, germane to the subject matter for which the assistive system is being used to benefit the participant, the engagement engine configured to use a combination of the application-specific content and the trusting relationship content to cause the avatar to interact with the participant in a manner that draws participant input from the participant to the engagement engine via the avatar due to the trusting relationship.

2. The assistive system of claim 1, wherein the trusting relationship dataset includes or is developed as a function of recorded information of interactions between the engagement engine, via the avatar, and the participant.

3. The assistive system of claim 1, wherein the trusting relationship dataset is:
a) accessible by a caregiver,
b) recorded information developed as a result of interactions between the avatar, participant, and optionally a third party, or
a) and b).

4. The assistive system of claim 1, wherein the engagement engine is configured to identify an inconsistency between a response from the participant, in reaction to a participant-focused question or command from the avatar, and an expected response based on:
content in the trusting relationship dataset; and
content in the application-specific database.

5. The assistive system of claim 1, wherein the engagement engine is further configured to recognize reduced intelligibility of a response from the participant, in reaction to a participant-focused question or command from the avatar, compared to intelligibility of a past response to the same or similar participant-focused question or command.

6. The assistive system of claim 1, wherein the avatar is a likeness of a character human, animal, or fictional creature.

7. The assistive system of claim 1, wherein the trusting relationship dataset includes content that is supplied by historical information associated with the participant or a person or organization associated with the participant or captured through interaction of the participant and the avatar, and wherein the content includes at least a subset of:
personal information of the participant;
physiological information of the participant;
psychological information of the participant;
personality information of the participant;
sociocultural information of the participant;
behavioral information of the participant;
cognitive information of the participant;
emotional information of the participant;
personal information of a person trusted by the participant or a person known by the participant;
common life experiences;
common connections to the person;
human relationship connections;
experiences in common with the person; and
interests of the participant or the person trusted by the participant, the interests including at least one of: entertainment, politics, vocation, avocation, and religion.

8. The assistive system according to claim 1, wherein the application-specific database includes content that is at least a subset of:
demographic information;
financial information;
education, vocation and occupation and professional information;
health and mental information;
preferences and interests; and
location or regional culture information.

9. The assistive system of claim 1, wherein the engagement engine is further configured to be activated by the participant or a third party.

10. The assistive system of claim 1, wherein the engagement engine is further configured to employ artificial intelligence (AI), machine learning, or combination thereof.

11. The assistive system of claim 1, further comprising a human-machine interface configured to represent the avatar to the participant and to capture interactions between the participant and the engagement engine.

12. The assistive system of claim 11, wherein at least one input to the human-machine interface represents an active state of the participant.

13. The assistive system of claim 11, wherein the engagement engine is further configured to interpret at least one input to the human-machine interface from the participant as an age-related cognitive disorder, a traumatic brain injury, a cognitive change at any point in time relative to an earlier point in time that is earlier relative to a current time at which the at least one input is input to the assistive system, or an earlier average level of cognitive function or learning performance, the earlier average level of cognitive function or learning performance being earlier relative to the current time.

14. The assistive system of claim 11, wherein the engagement engine is further configured to interpret at least one input to the human-machine interface from the participant based on a level of achievement of the participant within an application the assistive system is being used to benefit the participant, the level of achievement associated with educational achievement or training achievement.

15. The assistive system of claim 14, wherein the application the assistive system is being used to benefit the participant is selected from a group including: medical evaluation, psychological evaluation, sales training and education, training, education, relationship training, school testing, proxy for third party, interview, and drug evaluation.

16. A method for diagnosing, monitoring, and improving cognitive health issues in a participant using an avatar, the computer-implemented method comprising:
collecting real-time data on the participant's behavior and cognitive responses through interactive sessions with the avatar used;
analyzing the real-time data collected, the analyzing using artificial intelligence (AI) computer-implemented methods, the AI computer-implemented methods including machine learning and anomaly detection techniques used to identify abnormalities, assess cognitive function, and provide cognitive exercises;
dynamically generating and conducting cognitive tests and exercises during the interactive sessions based on the participant's inputs and performance to gather additional diagnostic and therapeutic data; and
providing comprehensive diagnostic and therapeutic information, including visualizations and trend analyses, to healthcare providers for timely intervention, personalized treatment, and cognitive recovery plans for the participant.
